(19) United States
(12) Patent Application Publication
(10) Pub. No.: US 2020/0305720 A1
(43) Pub. Date: Oct. 1, 2020

(54) IMAGING DEVICE

(71) Applicant: Noritaka AKITA, Kyoto-shi, Kyoto (JP)

(72) Inventor: Noritaka AKITA, Kyoto-shi, Kyoto (JP)

(21) Appl. No.: 16/310,886

(22) PCT Filed: Feb. 24, 2017
(86) PCT No.: PCT/JP2017/007180

§ 371 Date: Dec. 18, 2018

(30) Foreign Application Priority Data
Jun. 30, 2016 (JP) 2016-129670

(51) Int. Cl. A61B 5/00

(52) U.S. Cl. CPC A61B 5/0071; A61B 2560/0214; A61B 5/4887

(57) ABSTRACT

An auxiliary excitation light source unit 50 is a portable one having a size capable of being held by one hand. The auxiliary excitation light source unit 50 includes an auxiliary excitation light source 51 including a high-power LED in a casing 57. The auxiliary excitation light source 51 emits near-infrared light that is excitation light adapted for exciting indocyanine green and whose wavelength is 760 nm. In front of the auxiliary excitation light source 51, a lowpass filter 53 for blocking light having a wavelength corresponding to the wavelength of fluorescence emitted from indocyanine green is disposed.

### TECHNICAL FIELD

This invention relates to an imaging apparatus adapted to irradiate a fluorescent dye administered into the body of a subject with excitation light, and to image fluorescence emitted from the fluorescent dye.

### BACKGROUND ART

In the past, as a method for visualizing blood vessels, lymphatic vessels, lymph nodes, and the like inside a living body in surgery, which are difficult to visually recognize, a dye method and an R.I. method that preliminarily injects a radioactive isotope have been utilized. On the other hand, in recent years, a method called near-infrared fluorescence imaging has been utilized for angiography in surgery. In the near-infrared fluorescence imaging, indocyanine green (ICG) as a fluorescent dye is injected by an injector or the like and thereby administered into an affected area. Then, when irradiating the indocyanine green with near-infrared light having a wavelength of approximately 600 to 850 nm as excitation light, the indocyanine green emits near-infrared fluorescence having a wavelength of approximately 750 to 900 nm. The fluorescence is imaged by an imaging device capable of detecting near-infrared light, and the resulting image is displayed on a display unit such as a liquid crystal display panel. The near-infrared fluorescence imaging makes it possible to observe blood vessels, lymphatic vessels, and the like present at a depth of approximately 20 mm or less from the surface of a body.

Also, in recent years, a method that fluorescently labels a neoplasm to utilize it for surgery navigation has been attracting attention. As a fluorescence labeling agent for fluorescently labeling neoplasms, 5-aminolevulinic acid (5-ALA) is used. When administering 5-aminolevulinic acid (hereinafter referred to as “5-ALA” when abbreviated) into a subject, 5-ALA is metabolized into PpIX (protoporphyrin IX) that is a fluorescent dye. Note that PpIX preferentially accumulates in cancer cells. Then, when irradiating PpIX as a metabolite of 5-ALA with visible light having a wavelength of approximately 410 nm, red visible light having a wavelength of approximately 630 nm is emitted from PpIX as fluorescence. Observing the fluorescence from PpIX makes it possible to confirm the cancer cells as in the case of indocyanine green.

Patent Literature 1 discloses a data collection method that compares a near-infrared fluorescence intensity distribution image obtained by irradiating a suspected body organ administered with indocyanine green with excitation light from indocyanine green and a cancer lesion distribution image obtained by making X-rays, nuclear magnetic resonance or an ultrasonic wave act on the suspected organ before the indocyanine green administration with each other, and as cancer sub-lesion region data, collects data on a region that is detected in the near-infrared fluorescence intensity distribution image but is not detected in the cancer lesion distribution image.

Also, Patent Literature 2 discloses an imaging apparatus including an illumination/imaging unit that, with use of the illumination/imaging unit in which a camera, an infrared light source, and a visible light source are integrated, irradiates a subject with infrared light and visible light and performs imaging by the camera. In addition, the illumination/imaging unit is movably supported by an arm. Arranging the illumination/imaging unit with the unit facing the subject simultaneously makes it possible to irradiate an arbitrary area of the subject with the infrared light and the visible light and to perform imaging by the camera.

### CITATION LIST

### Patent Literature

### [Patent Literature 1]

International Patent Publication No. 2009/139466

### [Patent Literature 2]

International Patent Publication No. 2015/092882

### SUMMARY OF INVENTION

### Technical Problem

For example, when performing breast cancer surgery in breast surgery utilizing indocyanine green, it is necessary to quickly identify the position of a sentinel lymph node. The sentinel lymph node is a lymph node that is first reached by cancer cells via lymph flow. If no cancer cells are found in the sentinel lymph node, it can be determined that the breast cancer has not metastasized to any other lymph node.

When performing a biopsy on the sentinel lymph node in the breast surgery, it is required to visualize indocyanine green accumulated in the sentinel lymph node with high visibility and high sensitivity. However, the sentinel lymph node in a breast is different among individuals in terms of the level of fat, a depth from the surface of a body, and the like, and therefore near-infrared fluorescence from the indocyanine green may be appropriately visually unrecognizable.

That is, as described in Patent Literature 2, in the configuration in which the infrared light source for exciting indocyanine green and the camera are integrated with each other, although there is the advantage of being able to make the irradiation direction with the infrared light and an imaging direction coincide with each other, it is necessary to set the distance (working distance) between an affected area and the camera to a few tens of centimeters or more for reasons such as the need to image a surrounding region including an affected area of the subject by the camera. For this reason, it may be impossible to make the infrared light from the infrared light source reach the affected area with sufficient excitation light energy density for indocyanine green. In such a case, near-infrared fluorescence from the indocyanine green cannot be appropriately visually recognized.

For example, in the case of a single point light source, the energy density of light is inversely proportional to the square of the distance. For this reason, in order to increase the energy density of the infrared light reaching the affected area of the subject while keeping a constant distance between the subject and the infrared light source, a high power light source or a number of light sources are required to obtain a light amount enough to compensate for attenuation caused by distance. This causes not only problems of complicating the apparatus and increasing weight but also problems of increasing the cost of the apparatus and requiring a cooling mechanism in association with an increase in the calorific value of the light source or light sources.

This invention has been made in order to solve the above problems, and intends to provide an imaging apparatus that, despite simplicity and low cost, can efficiently irradiate a subject with excitation light and acquire a clear fluorescence image.

### Solution to Problem

A first aspect of this invention includes: an illumination/imaging unit, the illumination/imaging unit including an excitation light source for irradiating the subject with excitation light adapted for exciting a fluorescent dye administered into a subject, and a camera for imaging fluorescence emitted from the fluorescent dye that is irradiated with the excitation light to acquires a fluorescence image; and a portable auxiliary excitation light source unit for irradiating the subject with the excitation light for exciting the fluorescent dye administered into the subject from a position adjacent to the subject.

In a second aspect of this invention according to the first aspect, the auxiliary excitation light source unit includes a light source and a battery adapted for lighting the light source.

In a third aspect of this invention according to the second aspect, the light source is an LED, and the battery is connected to the LED via a constant current circuit.

In a fourth aspect of this invention according to the first aspect, the illumination/imaging unit is supported by a support member.

### Advantageous Effects of Invention

According to the first aspect of this invention, by irradiating the subject with the excitation light from a short distance utilizing the auxiliary excitation light source unit in addition to the excitation light from the excitation light source in the illumination/imaging unit, excitation light can be efficiently emitted to the subject and a clear fluorescence image can be acquired.

According to the second aspect of this invention, since the light source can be lit by the battery, the need for wiring for supplying electricity is eliminated. For this reason, the auxiliary excitation light source unit can be covered with a sterile drape or the like, and a clean state can be very easily kept.

According to the third aspect of this invention, the illumination intensity of the excitation light emitted from the LED supplied with electricity from the battery can be prevented from gradually decreasing.

According to the fourth aspect of this invention, a fluorescence image generated by the action of the excitation light from the excitation light source in the illumination/imaging unit supported by the support member and the excitation light from the auxiliary excitation light source unit can be efficiently imaged by an imaging part in the illumination/imaging unit.

### BRIEF DESCRIPTION OF DRAWINGS

FIG. 1 is a perspective view illustrating the apparatus body of the imaging apparatus according to this invention.

FIG. 2 is a side view illustrating the apparatus body of the imaging apparatus according to this invention.

FIG. 3 is a plan view illustrating the apparatus body of the imaging apparatus according to this invention.

FIG. 4 is a perspective view of an illumination/imaging unit 12.

FIG. 5 is a perspective view of an auxiliary excitation light source unit 50.

FIG. 6 is a schematic cross-sectional view of the auxiliary excitation light source unit 50.

FIG. 7A is a graph illustrating the relationship between the illumination intensity of near-infrared light emitted from an auxiliary excitation light source 51 and time.

FIG. 7B is a graph illustrating the relationship between the illumination intensity of the near-infrared light emitted from the auxiliary excitation light source 51 and time.

FIG. 8 is an explanatory view illustrating how a fluorescence image is imaged utilizing the imaging apparatus according to this invention.

### DESCRIPTION OF EMBODIMENTS

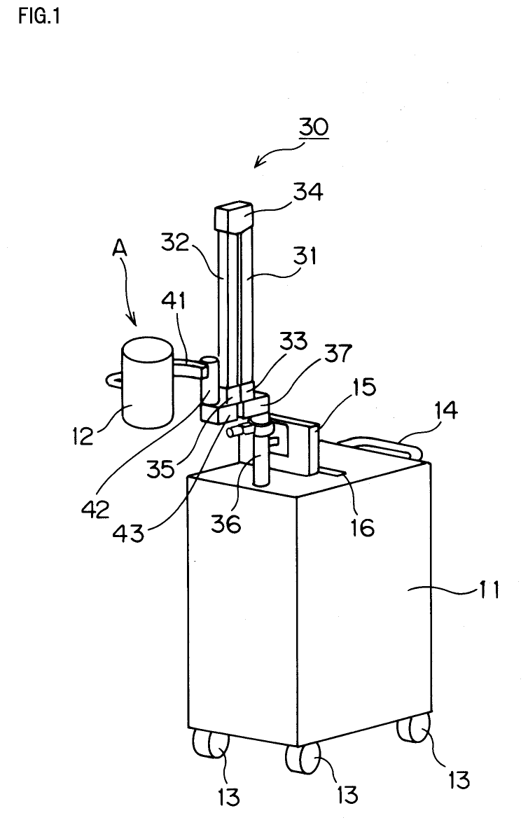

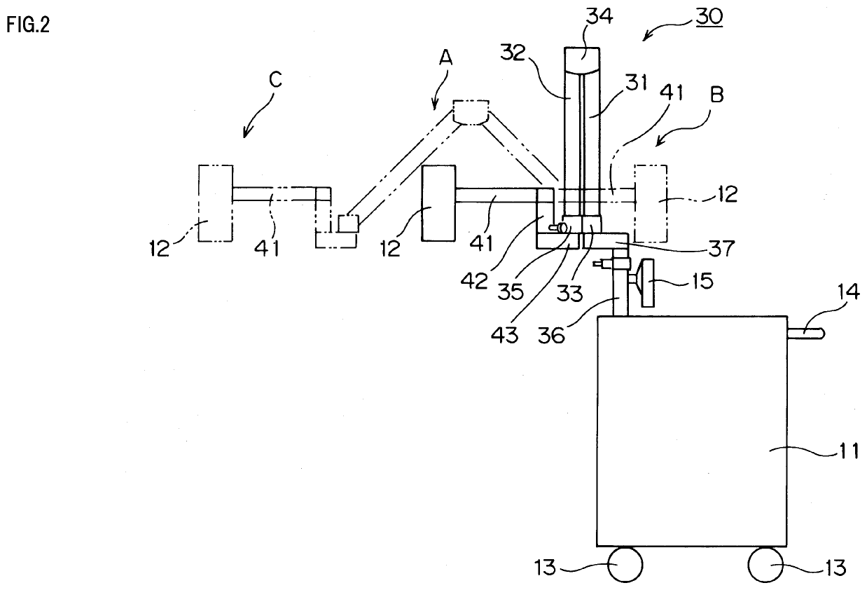

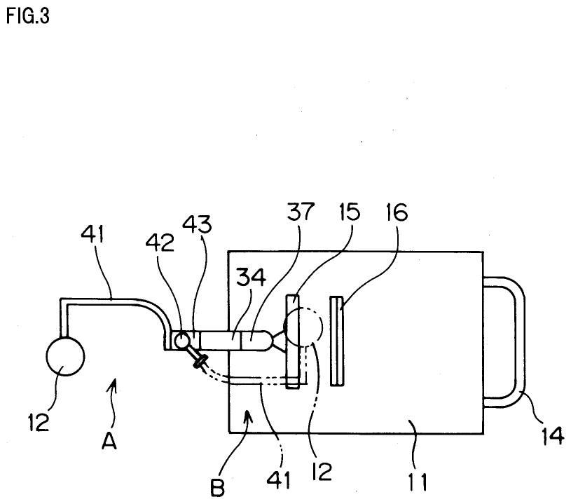

In the following, an embodiment of this invention will be described on the basis of the drawings. The imaging apparatus according to this invention is configured to include: an apparatus body having an illumination/imaging unit 12; and an auxiliary excitation light source unit 50. First, the configuration of the apparatus body will be described. FIG. 1 is a perspective view illustrating the apparatus body of the imaging apparatus according to this invention. FIG. 2 is a side view illustrating the apparatus body of the imaging apparatus according to this invention. FIG. 3 is a plan view illustrating the apparatus body of the imaging apparatus according to this invention.

The apparatus body of the imaging apparatus according to this invention is one for irradiating indocyanine green as a fluorescent dye injected into the body of a subject with excitation light and imaging fluorescence emitted from the indocyanine green, and includes: a cart 11 provided with four wheels 13; an arm mechanism 30 disposed on the upper surface of the cart 11 and near the front in the traveling direction of the cart 11 (left direction in FIG. 2 and FIG. 3); the illumination/imaging unit 12 disposed on the arm mechanism 30 via a sub-arm 41; and a monitor 15. At the back in the traveling direction of the cart 11, a handle 14 that is used when moving the cart 11 is annexed. In addition, on the upper surface of the cart 11, a concave part 16 for placing a remote controller for remotely controlling the imaging apparatus is formed.

The above-described arm mechanism 30 is disposed on the front side in the traveling direction of the cart 11. The arm mechanism 30 includes a first arm member 31 connected via a hinge part 33 to a support part 37 disposed on a support post 36 provided upright on the front side in the traveling direction of the cart 11. The first arm member 31 is swingable with respect to the cart 11 via the support post 36 and the support part 37 by the action of the hinge part 33. In addition, the above-described monitor 15 is annexed to the support post 36.

The upper end of the first arm member 31 is connected with a second arm member 32 by a hinge part 34. The second arm member 32 is swingable with respect to the first arm member 31 by the action of the hinge part 34. For this reason, the first arm member 31 and the second arm member 32 are capable of taking an imaging position where the first arm member 31 and the second arm member 32 are spread at a predetermined angle around the hinge part 34 as a connecting part between the first arm member 31 and the second arm member 32 as indicated by virtual lines marked with the symbol C in FIG. 2 and a standby position where the first arm member 31 and the second arm member 32 are adjacent as indicated by solid lines marked with the symbol A in FIG. 1 to FIG. 3.

The lower end of the second arm member 32 is connected with a support part 43 by a hinge part 35. The support part 43 is swingable with respect to the second arm member 32 by the action of the hinge part 35. The support part 43 supports a rotary shaft 42. In addition, the sub-arm 41 supporting the illumination/imaging unit 12 rotationally moves around the rotary shaft 42 disposed at the fore end of the second arm member 32. For this reason, on the basis of the rotational movement of the sub-arm 41, the illumination/imaging unit 12 moves between a position on the front side in the traveling direction of the cart 11 with respect to the arm mechanism 30, which is for taking the imaging position or the standby position as indicated by the solid lines marked with the symbol A in FIG. 1 to FIG. 3 or as indicated by the virtual lines marked with the symbol C in FIG. 2 and a position on the back side in the traveling direction of the cart 11 with respect to the arm mechanism 30 as indicated by the virtual lines marked with the symbol B in FIG. 2 and FIG. 3, which is a position to move the cart 11.

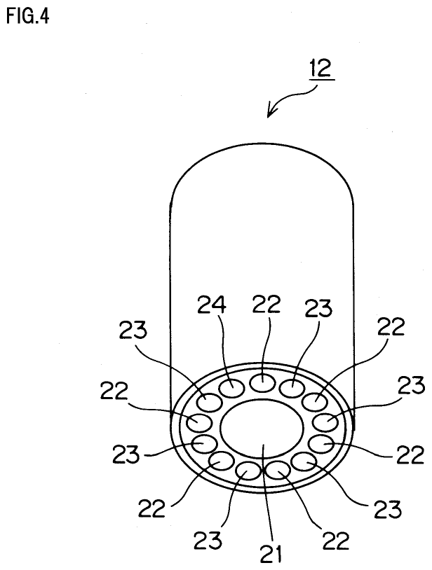

FIG. 4 is a perspective view of the illumination/imaging unit 12.

The illumination/imaging unit 12 includes: a camera 21 having multiple imaging devices capable of detecting near-infrared light and visible light; a visible light source 22 including six LEDs disposed in the outer circumferential part of the camera 21; an excitation light source 23 including six LEDs; and a confirmation light source 24 including one LED. The visible light source 22 emits visible light. The excitation light source 23 emits near-infrared light that is excitation light adapted for exciting indocyanine green and whose wavelength is 760 nm. Further, the confirmation light source 24 emits near-infrared light whose wavelength is 810 nm approximate to the wavelength of fluorescence emitted from indocyanine green. Note that the wavelength from the excitation light source 23 is not limited to 760 nm but only has to be a wavelength capable of exciting indocyanine green. The wavelength from the confirmation light source 24 is not limited to 810 nm but may be the wavelength emitted by indocyanine green or longer.

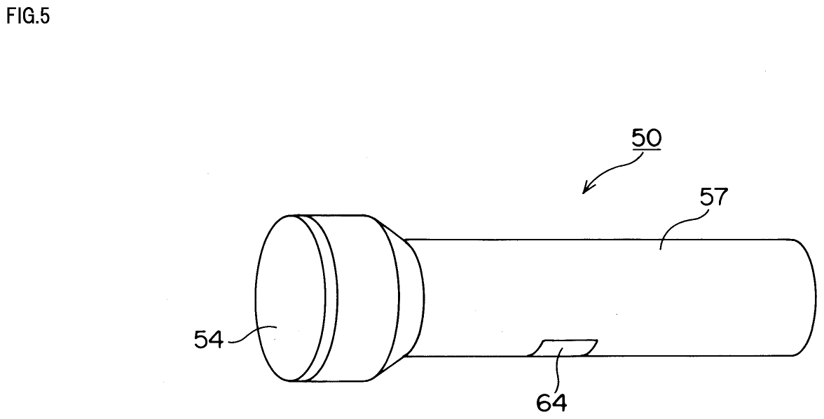

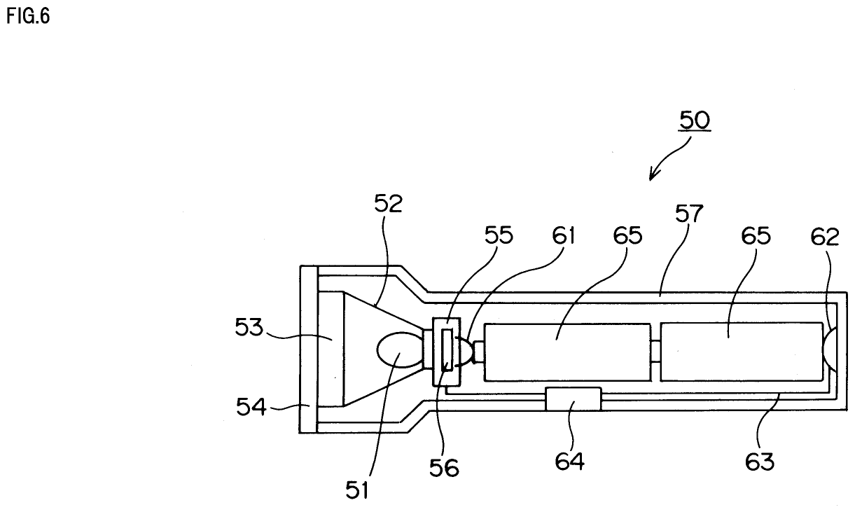

Next, the configuration of the auxiliary excitation light source unit 50 will be described. FIG. 5 is a perspective view of the auxiliary excitation light source unit 50. Also, FIG. 6 is a schematic cross-sectional view of the auxiliary excitation light source unit 50.

The auxiliary excitation light source unit 50 is a portable one having a size capable of being held by one hand. The auxiliary excitation light source unit 50 includes an auxiliary excitation light source 51 including a high-power LED in a casing 57. The auxiliary excitation light source 51 emits near-infrared light that is excitation light adapted for exciting indocyanine green as a fluorescent dye and whose wavelength is 760 nm, as with the excitation light source 23 in the illumination/imaging unit 12. Around the auxiliary excitation light source 51, a reflective mirror 52 is disposed. Also, in front of the auxiliary excitation light source 51, a lowpass filter 53 for blocking light having a wavelength corresponding to the wavelength of the fluorescence emitted from indocyanine green is disposed. The front of the lowpass filter 53 in the casing 57 is covered with an acrylic protective cover 54.

The auxiliary excitation light source 51 is connected to a light source drive board 55. The light source drive board 55 is connected via a terminal 61 to the plus side of two batteries 65 disposed in series in the casing 57, and also connected to the minus side of the batteries 65 via a terminal 62, a conductive wire 63, and a pushbutton switch 64. The light source drive board 55 includes a constant current circuit 56. That is, the batteries 65 are connected via the constant current circuit 56 to the auxiliary excitation light source 51 including the LED.

Note that as described above, by employing the configuration incorporating the batteries 65 as the auxiliary excitation light source unit 50, the need for wiring for supplying electricity is eliminated. For this reason, the auxiliary excitation light source unit 50 can be covered with a sterile drape or the like, and a clean state can be very easily kept. In this case, the auxiliary excitation light source 51 is configured to be lit and extinguished with the pushbutton switch 64, and therefore the lighting and extinguishing operations can be easily performed via the sterile drape, or the like.

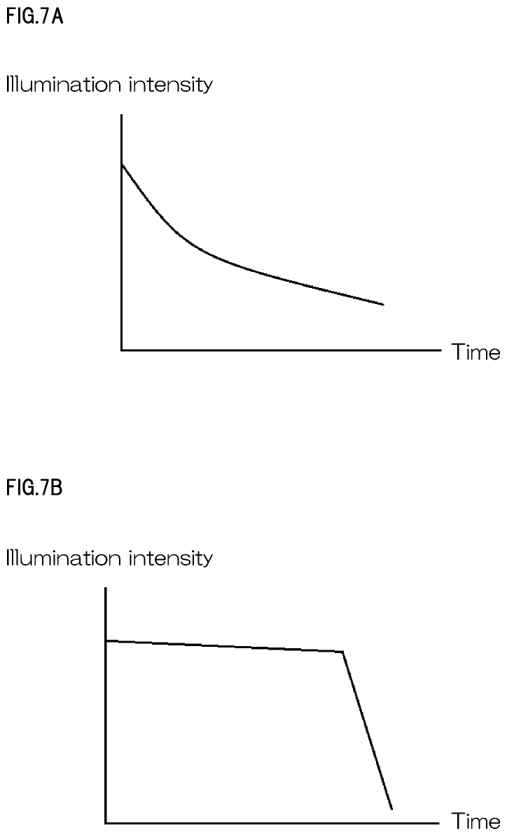

FIG. 7A and FIG. 7B are graphs illustrating the relationship between the illumination intensity of the near-infrared light emitted from the auxiliary excitation light source 51 and time. Note that FIG. 7A illustrates the case where the batteries 65 and the auxiliary excitation light source 51 are directly connected, and FIG. 7B illustrates the case where the batteries 65 are connected to the auxiliary excitation light source 51 via the constant current circuit 56.

In the case where the batteries 65 and the auxiliary excitation light source 51 are directly connected, current flowing through the auxiliary excitation light source 51 gradually decreases as time goes on because of a rise in the temperature of the auxiliary excitation light source 51 including the high-power LED, and the like, and thereby the illumination intensity of the excitation light emitted from the auxiliary excitation light source 51 is gradually decreased. On the other hand, in the case where the batteries 65 are connected to the auxiliary excitation light source 51 via the constant current circuit 56, the current flowing through the auxiliary excitation light source 51 can be kept constant, and the illumination intensity of the excitation light emitted from the auxiliary excitation light source 51 can be kept at constant illumination intensity for a certain period of time.

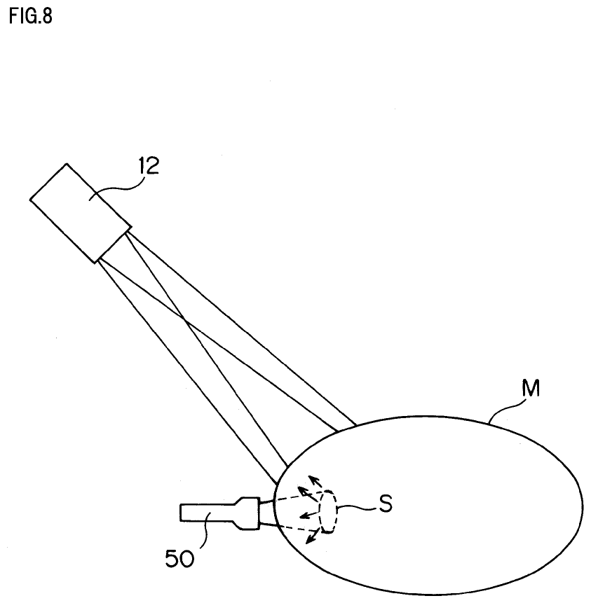

Next, operation when performing surgery using the imaging apparatus according to this invention will be described. FIG. 8 is an explanatory view illustrating how a fluorescence image is imaged utilizing the imaging apparatus according to this invention. In addition, in the following description, described is a case where surgery is performed on a subject (patient) M.

When performing surgery using the imaging apparatus according to this invention, first, the confirmation light source 24 in the illumination/imaging unit 12 is lit, and also an image at the time is imaged by the camera 21. From the confirmation light source 24, the near-infrared light having a wavelength of 810 nm approximate to the wavelength of the fluorescence emitted from indocyanine green is emitted. The near-infrared light cannot be recognized by human eyes. Meanwhile, when emitting the near-infrared light having a wavelength of 810 nm from the confirmation light source 24 and imaging the image of a corresponding irradiation area by the camera 21, and when the camera 21 normally operates, the image of the area irradiated with the near-infrared light is imaged by the camera 21, and the image is displayed on the display unit of which illustration is omitted. This makes it possible to easily confirm the operation of the camera 21.

After that, the subject M is injected with indocyanine green by a syringe. Then, the excitation light source 23 in the illumination/imaging unit 12 irradiates the affected area S of the subject M with the near-infrared light and also the visible light source 22 irradiates the affected area S of the subject M with the visible light. In addition, as the near-infrared light emitted from the excitation light source 23, as described above, the 760 nm near-infrared light acting as excitation light for allowing indocyanine green to emit fluorescence is employed. This allows the indocyanine green to emit fluorescence having a peak at approximately 800 nm in the near-infrared region.

Subsequently, the affected area S and its surroundings of the subject M are imaged by the camera 21 in the illumination/imaging unit 12. The camera 21 is capable of detecting near-infrared light and visible light. A near-infrared image and a visible image imaged by the camera 21 are converted by an image processing unit into pieces of image data capable of displaying the near-infrared image and the visible image on the display unit, and displayed on the unillustrated display unit such as a liquid crystal display panel. Further, as needed, the image processing unit utilizes the near-infrared image data and the visible image data to generate a synthetic image in which the visible image and the near-infrared image are synthesized.

At this time, between the illumination/imaging unit 12 and the subject M, a distance (working distance) of a few tens of centimeters or more exists. Meanwhile, a sentinel lymph node as the affected area S is different among individuals in terms of the level of fat, a depth from the surface of a body, and the like. For this reason, the near-infrared fluorescence from the indocyanine green may be appropriately visually unrecognizable. In such a case, as illustrated in FIG. 8, the auxiliary excitation light source unit 50 is used.

The auxiliary excitation light source unit 50 is preliminarily covered with a sterile drape. Then, an operator holds the portable auxiliary excitation light source unit 50 in an operator's hand, and as illustrated in FIG. 8, irradiates the affected area S with the excitation light from a position near the body surface of the subject M.

As described above, the energy density of light is inversely proportional to the square of the distance. For this reason, if the distance between the excitation light source 23 in the illumination/imaging unit 12 and the subject M is set to 70 centimeters and the distance between the protective cover 54 of the auxiliary excitation light source 51 in the auxiliary excitation light source unit 50 and the body surface of the subject is set to 10 centimeters, and if the emission intensities of the excitation lights are the same, by utilizing the auxiliary excitation light source unit 50, the subject M is irradiated with excitation light having approximately 50-fold intensity. For this reason, even when using the small-size, light-weight, and portable auxiliary excitation light source unit 50 as illustrated in FIG. 8, an excitation effect several times in the case of using the excitation light source 23 in the illumination/imaging unit 12 can be obtained. Also, when using the portable auxiliary excitation light source unit 50, the excitation light can be emitted from the most suitable position and direction for the affected area S. Therefore, even the affected area S having poor visibility, such as a sentinel lymph node, can be clearly imaged. In addition, as an approach distance, the distance between the protective cover 54 of the auxiliary excitation light source 51 and the body surface of the subject is set to a distance that is 30 cm or less and avoids contact with the subject M.

In this case, since in the auxiliary excitation light source unit 50, the batteries 65 are connected via the constant current circuit 56 to the auxiliary excitation light source 51 including the high-power LED, the current flowing through the auxiliary excitation light source 51 can be kept constant. This makes it possible to keep the illumination intensity of the excitation light emitted from the auxiliary excitation light source 51 at constant illumination intensity for a certain period of time.

As described above, in the imaging apparatus according to this embodiment, while illuminating the affected area S with the excitation light and imaging the affected area S by the illumination/imaging unit 12 supported by the arm mechanism 30 as a support member, the auxiliary excitation light source unit 50 can irradiate the affected area S with the excitation light from a position near the affected area S of the subject M and therefore a very clear excitation image of indocyanine green can be efficiently obtained.

In addition, in the above-described embodiment, described is the case where by using indocyanine green as a material containing a fluorescent dye and irradiating the indocyanine green with near-infrared light of approximately 600 nm to 850 nm as excitation light, the fluorescent having a peak at approximately 810 nm in the near-infrared region is emitted from the indocyanine green; however, light other than near-infrared light may be used.

Also, in place of using indocyanine green as a fluorescent dye, another fluorescent dye such as 5-ALA described above may be used.

### REFERENCE SIGNS LIST

12 Illumination/imaging unit
21 Camera
22 Visible light source
23 Excitation light source
24 Confirmation light source
30 Arm mechanism
50 Auxiliary excitation light source unit
51 Auxiliary excitation light source
52 Reflective mirror
53 Lowpass filter
54 Protective cover
55 Light source drive board
56 Constant current circuit
65 Batteries
M Subject
S Affected area

1. An imaging apparatus comprising:
an illumination/imaging unit, the illumination/imaging unit including
an excitation light source for irradiating the subject with excitation light adapted for exciting a fluorescent dye administered into a subject, and
a camera for imaging fluorescence emitted from the fluorescent dye that is irradiated with the excitation light to acquire a fluorescence image; and
a portable auxiliary excitation light source unit for irradiating the subject with the excitation light adapted for exciting the fluorescent dye administered into the subject from a position adjacent to the subject.

2. The imaging apparatus according to claim 1, wherein
the auxiliary excitation light source unit comprises a light source and a battery adapted for lighting the light source.

3. The imaging apparatus according to claim 2, wherein
the light source is an LED, and
the battery is connected to the LED via a constant current circuit.

4. The imaging apparatus according to claim 1, wherein
the illumination/imaging unit is supported by a support member.
